(19) United States
(12) Patent Application Publication
(10) Pub. No.: US 2009/0093448 A1
(43) Pub. Date: Apr. 9, 2009

(54) 19-NOR-PROGESTERONE FOR CONTRACEPTION

(75) Inventors: TAMARA PFAFF, DUSSELDORF (DE); THOMAS OTTEN, ROETGEN (DE); ULRICH JAHNEL, REMSCHEID (DE)

Correspondence Address: NORRIS, MCLAUGHLIN & MARCUS, PA, 875 THIRD AVENUE, 18TH FLOOR, NEW YORK, NY, 10022, US

(73) Assignee: GRUNENTHAL GMBH, AACHEN (DE)

(21) Appl. No.: 12/244,480

(22) Filed: Oct. 2, 2008

(30) Foreign Application Priority Data
Oct. 4, 2007 (DE) 10 2007 047 608.8

(51) Int. Cl. A61K 31/575; C07J 9/00; A61P 15/00

(52) U.S. Cl. 514/177; 552/555

(57) ABSTRACT

The invention relates to compounds of the general formula (I)

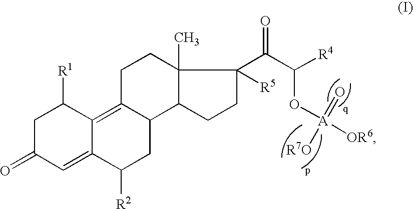

to a method for the production thereof, to pharmaceutical, cosmetic compositions or dosage forms which contain these compounds, and to a method for contraception by administration of these compounds.

The invention relates to compounds of the general formula (I)

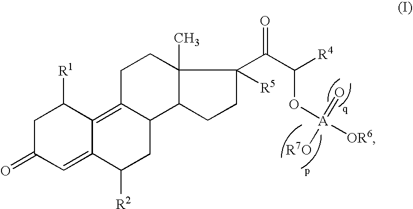

to a method for the production thereof, to pharmaceutical, cosmetic compositions or dosage forms which contain these compounds, to a method for contraception by administration of these pharmaceutical compositions, and to the use of these compounds for producing medicaments.

One of the most widely used methods of contraception is the administration or application of steroid hormones such as oestrogens in combination with gestagens. However, many of the currently used commercially obtainable hormonal contraceptives frequently exhibit side-effects which mean that taking of the contraceptive must be suspended, thus meaning that effective therapy or contraception is not longer ensured.

One object of the invention is to provide compounds which are suitable as pharmaceutical active ingredients and exhibit advantages over conventional pharmaceutical active ingredients. The pharmaceutical active ingredients should in particular be suitable for contraception.

This object is achieved by the subject matter of the claims.

It has surprisingly been found that the compounds of the general formula (I) exhibit an affinity for the human progesterone receptor and are therefore in particular suitable as pharmaceutical active ingredients, for example for hormone replacement therapy or for contraception.

The present invention provides a compound of the general formula (I)

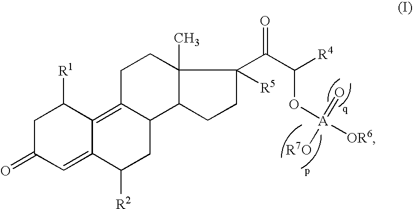

wherein
A is either a sulfur atom, p=0 and q=1 or 2; or is a phosphorus atom and p=1 and q=0 or 1;
R¹ and R² are in each case mutually independently —H, —OH or —OCO—R³;
R³, R⁴, R⁵, R⁶ and R⁷ are in each case mutually independently in each case —H or a linear or branched hydrocarbon residue with 1 to 12 carbon atoms, wherein the hydrocarbon residue is unsubstituted or is substituted with optionally 1, 2, 3, 4 or 5 substituents mutually independently selected from the group consisting of —F, —Cl, —Br, —I and —OH;
   or the pharmaceutically acceptable salts and/or solvates thereof.

Where, for the purposes the description, reference is made to compounds of the general formula (I), the pharmaceutically acceptable salts or solvates are also included, even if these are not in each case explicitly mentioned.

If A is a phosphorus atom, then p=1 and q=0 or 1. If q=0, then the phosphorus atom has the oxidation number III and the compound of the general formula (I) is a phosphonate. For clarity's sake, the tautomeric form is not taken into account in the general formula (I), but a person skilled in the art will recognise that, depending on the meaning of R⁶ and R⁷, the tautomeric form may also be present. If q=1, the phosphorus atom has the oxidation number IV and the compound of the general formula (I) is a phosphate.

If A is a sulfur atom, then p=0 and q=1 or 2. If q=1, then the sulfur atom has the oxidation number IV and the compound of the general formula (I) is a sulfite. If q=2, then the sulfur atom has the oxidation number VI and the compound of the general formula (I) is a sulfate.

For the purposes of the description, a linear or branched hydrocarbon residue should be taken to mean an acyclic, saturated (=alkyl) or mono- or polyunsaturated (for example=alkenyl or alkynyl) hydrocarbon residue. If the hydrocarbon residue is unsaturated, it may comprise at least one double bond and/or a triple bond, preferably 1, 2 or 3 double bonds and/or triple bonds. Examples of suitable linear or branched, saturated or unsaturated hydrocarbon residues which may be mentioned are methyl, ethyl, n-propyl, iso-propyl, n-butyl, iso-butyl, 2-butyl, tert-butyl, n-pentyl, neo-pentyl, n-hexyl, n-heptyl, n-octyl, n-nonyl, n-decyl, ethenyl, 1-propenyl, 2-propenyl, 1-butenyl, 2-butenyl, 3-butenyl, 1-pentenyl, 2-pentenyl, 3-pentenyl, 4-pentenyl, hexenyl, —CH═CH—CH═CH—CH₃ and —CH₂—CH₂—CH═CH₂.

Preferred compounds of the general formula (I) are those wherein

A is a sulfur atom with p=0 and q=2;

R¹ and R² are in each case mutually independently —H, —OH or —OCO—R³; and

R⁴ and R⁵ are in each case mutually independently —C₁₋₆ alkyl, preferably methyl, and

R³, R⁶ and R⁷ in each case have the above-stated meaning;

in each case optionally in the form of corresponding pharmaceutically acceptable salts and/or corresponding solvates.

Preferred compounds are those of the general formula (I-A),

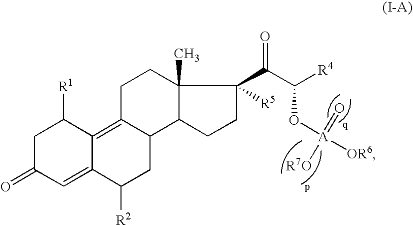

wherein p, q, R¹, R², R³, R⁴, R⁵, R⁶ and R⁷ in each case have the above-stated meaning; in each case optionally in the form of corresponding pharmaceutically acceptable salts and/or corresponding solvates.

Particularly preferred compounds are those of the general formula (I) or (I-A), wherein R¹ is —H; and the remaining residues have the above-stated meaning; in each case optionally in the form of corresponding pharmaceutically acceptable salts and/or corresponding solvates. Furthermore preferred compounds are those of the general formula (I) or (I-A), wherein R² is —H; and the remaining residues have the above-stated meaning; in each case optionally in the form of corresponding pharmaceutically acceptable salts and/or corresponding solvates. In particular, the following are preferred: compounds of the general formula (I) or (I-A), wherein both R¹ and R² is —H; and the remaining residues have the above-stated meaning; in each case optionally in the form of corresponding pharmaceutically acceptable salts and/or corresponding solvates.

Preferred compounds are also those of the general formula (I) or (I-A), wherein R⁴ is —C₁₋₆ alkyl, preferably methyl; and the remaining residues have the above-stated meaning; in each case optionally in the form of corresponding pharmaceutically acceptable salts and/or corresponding solvates. Furthermore preferred compounds are those of the general formula (I) or (I-A), wherein R⁵ is —C₁₋₆ alkyl, preferably methyl; and the remaining residues have the above-stated meaning; in each case optionally in the form of corresponding pharmaceutically acceptable salts and/or corresponding solvates. Compounds which are in particular preferred are those of the general formula (I) or (I-A), wherein both R⁴ and also R⁵ are in each case mutually independently —C₁₋₆ alkyl, preferably methyl; and the remaining residues have the above-stated meaning; in each case optionally in the form of corresponding pharmaceutically acceptable salts and/or corresponding solvates.

Furthermore preferred compounds are those of the general formula (I) or (I-A), wherein R¹ is —H and R² is —OH or —OCO—C₁₋₆ alkyl; or R¹ is —OH or —OCO—C₁₋₆ alkyl, and R² is —H; and the remaining residues have the above-stated meaning; in each case optionally in the form of corresponding pharmaceutically acceptable salts and/or corresponding solvates.

Furthermore preferred compounds are those of the general formula (I-B), (I-B′), (I-B″), (I-C), (I-C′) or (I-C″)

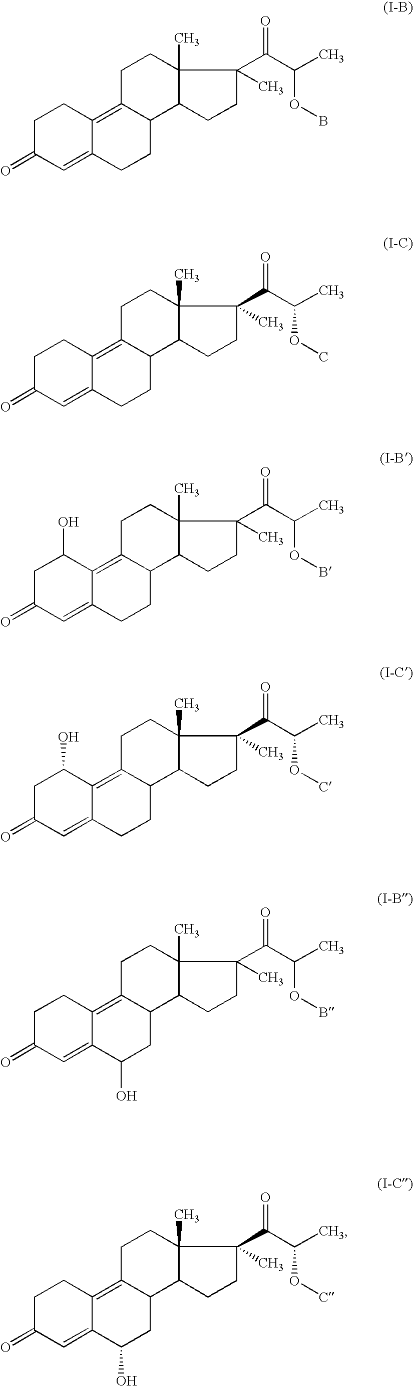

wherein B, B′, B″, C, C′ and C″ denote a residue which is selected from the group consisting of

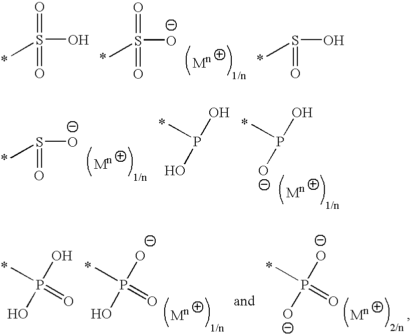

and wherein Mⁿ⁺ is a pharmaceutically acceptable cation with n=1, 2 or 3; in each case optionally in the form of corresponding pharmaceutically acceptable salts and/or corresponding solvates.

A pharmaceutically acceptable cation should be taken for the purposes of the description preferably to mean a cation which is monovalent (1 positive charge), divalent (2 positive charges) or trivalent (3 positive charges) and is in general physiologically safe. The cation is preferably derived from an organic or inorganic base.

In a preferred embodiment, the cation is a metal cation, preferably selected from the group consisting of cations of main group metals, in particular alkali metals and alkaline earth metals, and transition metals.

In another preferred embodiment, the cation is an organic cation, preferably a quaternary ammonium compound N⁺RR′R″R″′, wherein R, R′, R″ and R″′ are preferably mutually independently —H or —C₁-C₈ alkyl, in each case optionally substituted with an —OH residue or at least two of the residues R, R′, R″ and R″′ form a saturated, unsaturated or aromatic four-, five-, six- or seven-membered ring. Other organic cations which are derived from organic bases, are protonated forms of ammonia, ethylenediamine, ethanolamine, 1 H-imidazole, diethylamine, piperazine, Deanol, diethanolamine, pyrrolidine, betaine, 2-(diethylamino)ethanol, tromethamine, choline, morpholine, lysine, triethanolamine, L-arginine, N-methylglucamine, benethamine, benzathine, hydrabamine, etc.

Pharmaceutically acceptable cations which may be mentioned by way of example are H⁺, Li⁺, Na⁺, K⁺, Mg²⁺, Ca²⁺, Al³⁺, Mn²⁺, Fe²⁺, Fe³⁺, Co²⁺, Co³⁺, Ni²⁺, Cu⁺, Cu²⁺, Ag⁺, Zn²⁺, NH₄⁺, N(C₁-C₈-alkyl)₄⁺, triethanolammonium, tris-(hydroxymethyl)aminomethane⁺ and pyridinium⁺. With regard to further details, reference may for example be made to the full content of P. H. Stahl et al., Handbook of Pharmaceutical Salts, Properties, Selection and Use, Wiley-VCH.

Particularly preferred compounds are those selected from the group consisting of

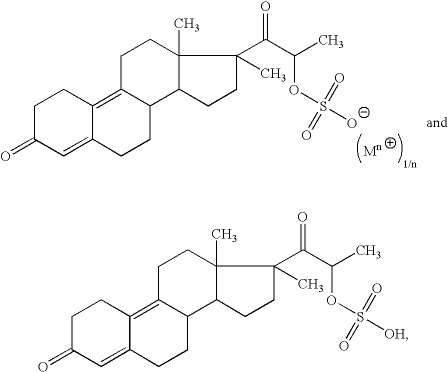

wherein Mⁿ⁺ is a pharmaceutically acceptable cation which is selected from the group consisting of Li⁺, Na⁺, K⁺, NH₄⁺, Ag⁺, Mg²⁺, Ca²⁺, Fe²⁺, Fe³⁺, Al³⁺ and pyridinium⁺; in each case optionally in the form of corresponding solvates.

A particularly preferred compound is one selected from the group consisting of

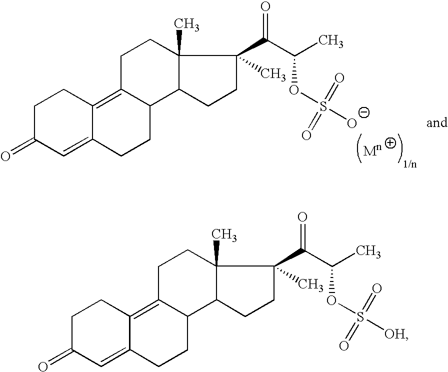

wherein Mⁿ⁺ is a pharmaceutically acceptable cation which is selected from the group consisting of Li⁺, Na⁺, K⁺, NH₄⁺, Ag⁺, Mg²⁺, Ca²⁺, Fe²⁺, Fe³⁺, Al³⁺ and pyridinium⁺; in case optionally in the form of corresponding solvates.

A particularly preferred compound is one selected from the group consisting of

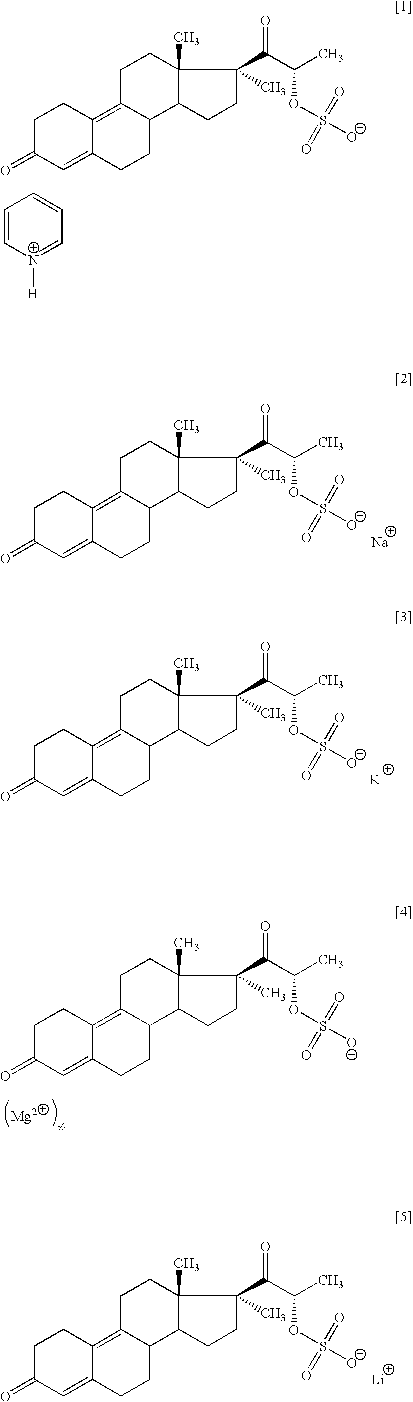

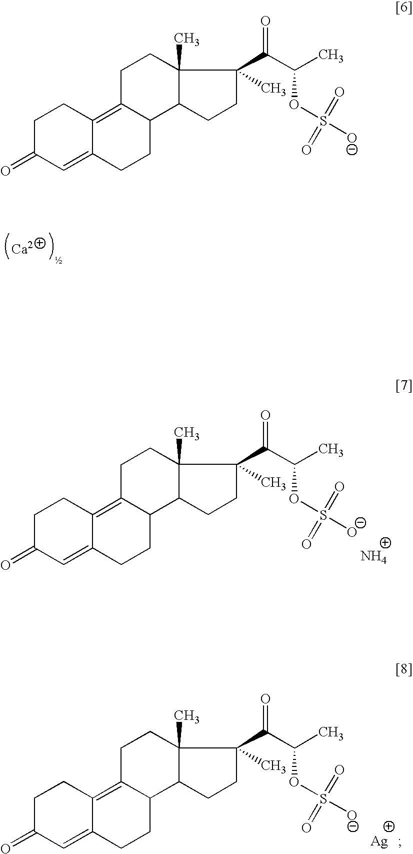

in each case optionally in the form of corresponding solvates.

The following compound is in particular preferred

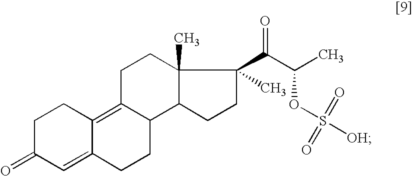

in each case optionally in the form of corresponding pharmaceutically acceptable salts and/or corresponding solvates.

The present invention also provides a method for producing compounds of the general formulae (I), (I-A), (I-B), (I-B′), (I-B″), (I-C), (I-C′) and (I-C″) comprising (a) reacting a compound of the general formula (II)

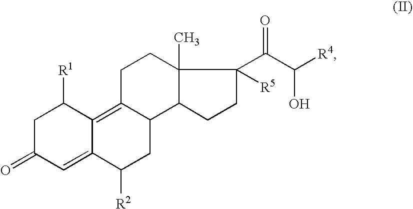

wherein R¹, R², R⁴ and R⁵ in each case have the above-stated meaning with a halide or anhydride of sulfurous acid, of sulfuric acid, of phosphorous acid or of phosphoric acid, preferably in a suitable solvent, preferably at a temperature of −20° C. to 100° C., more preferably at a temperature of 0° C. to 100° C., still more preferably at a temperature of 5° C. to 70° C. most preferably at a temperature of 10° C. to 50° C. and in particular at a temperature of 15° C. to 25° C.

In a preferred embodiment, the anhydride of sulfuric acid is complexed with pyridine {pyridine.SO₃}, with dimethylformamide {(HCON(CH₃)₂.SO₃}, with N-ethyldiisopropylamine {[(CH₃)2CH]₂NCH₂CH₃.SO₃}, with triethylamine {(CH₃CH₂)₃N.SO₃} or with trimethylamine {(CH₃)₃N.SO₃}. The anhydride of sulfuric acid is particularly preferably complexed with pyridine.

The method according to the invention preferably comprises as a further step (b) conversion of the product obtained in step (a) into a (another) pharmaceutically acceptable salt. Methods for converting a salt into another salt (metathesis) are known to a person skilled in the art.

The above-described reactions may in each case be performed under conventional conditions familiar to a person skilled in the art, for example with regard to pressure or the sequence of addition of the components. Optimum control of the method according to the respective conditions may optionally be established by a person skilled in the art by simple preliminary testing.

The present invention also provides compounds of the general formulae (I), (I-A), (I-B), (I-B′), (I-B″), (I-C), (I-C′) and (I-C″) (I-B), (I-B′), (I-B″), (I-C), (I-C′) or (I-C″) obtainable by the above-stated method.

The intermediate and final products obtained from the above-described reactions may in each case, if desired and/or necessary, be purified and/or isolated using conventional methods known to a person skilled in the art. Suitable purification methods are for example extraction methods and chromatographic methods.

All the above-described method steps and in each case also the purification and/or isolation of intermediate or final products may be performed in part or entirely under an inert gas atmosphere, preferably under a nitrogen atmosphere.

The compounds according to the invention of the general formula (I), (I-A), (I-B), (I-B′), (I-B″), (I-B″′), (I-C′), (I-C″) and (I-C″′), which are hereinafter denoted compounds of the general formula (I), and optionally in each case corresponding stereoisomers may be obtained using conventional methods known to the person skilled in the art in the form of corresponding salts, in particular in the form of corresponding physiologically acceptable salts.

The compounds according to the invention of the above-stated general formula (I) and optionally corresponding stereoisomers and in each case the pharmaceutically/physiologically acceptable salts thereof may be obtained using conventional methods known to a person skilled in the art also in the form of the solvates thereof, in particular in the form of the hydrates thereof.

The compounds according to the invention of the general formula (I) and optionally corresponding stereoisomers and in each case the corresponding pharmaceutically/physiologically acceptable salts and solvates appear to be toxicologically safe. Furthermore, these compounds may exhibit a longer half-life than for example trimegestone, for which reason these compounds are particularly suitable as pharmaceutical active ingredients in pharmaceutical compositions or for contraception.

In a preferred embodiment, the compounds according to the invention of the formula (I) exhibit a relative binding affinity for the human progesterone receptor of at least 10%, more preferably of at least 15%, still more preferably of at least 20%, most preferably of at least 25%, of at least 30%, of at least 35%, of at least 40%, of at least 45%, of at least 50% or of at least 55% and in particular of at least 60%, of at least 65%, of at least 70%, of at least 75%, of at least 80%, of at least 85%, of at least 90%, or of at least 95%, with progesterone being used as the reference substance for binding to the human progesterone receptor (=100% value). The above-stated binding affinity for the human progesterone receptor is here preferably determined according to EP-A 808 845 or as in I. Lacroix et al., Bioorganic & Medicinal Chemistry, 1999, 7, 2329-2341.

The present invention also provides a pharmaceutical composition containing at least one compound of the general formula (I), in each case optionally in the form of corresponding pharmaceutically acceptable salts and/or corresponding solvates. The pharmaceutical composition according to the invention may comprise one or more salts of one or more of these compounds.

The pharmaceutical composition preferably contains one or more pharmaceutically acceptable auxiliary substances. The quantity of the compound of the general formula (I) in the pharmaceutical composition according to the invention preferably amounts to at least 100 μg, more preferably to at least 200 μg, still more preferably to at least 300 μg, most preferably to at least 400 μg and in particular to at least 500 μg. In a preferred embodiment, the quantity of the compound of the general formula (I) in the pharmaceutical composition according to the invention is in the range from 500 μg to 3,000 μg, more preferably from 510 to 2,500 μg, still more preferably from 525 to 2,000 μg, most preferably from 550 to 1,500 μg and in particular from 600 to 900 μg. In another preferred embodiment, the quantity of the compound of the general formula (I) in the pharmaceutical composition according to the invention corresponds to an equivalent dose of trimegestone of at least 100 μg, more preferably of at least 200 μg, still more preferably of at least 300 μg, most preferably of at least 400 μg and in particular of at least 500 μg. In a preferred embodiment, the quantity of the compound of the general formula (I) in the pharmaceutical composition according to the invention corresponds to an equivalent dose of trimegestone in the range from 500 μg to 3,000 μg, more preferably from 510 to 2,500 μg, still more preferably from 525 to 2,000 μg, most preferably from 550 to 1,500 μg and in particular from 600 to 900 μg.

The equivalent dose of the compound of the general formula (I) in comparison with trimegestone is here selected such that the gestagenic activity corresponds that which would be brought about by the administration of the stated quantity of trimegestone. Suitable methods for determining the equivalent dose are known to a person skilled in the art.

The pharmaceutical composition preferably contains the at least one compound of the general formula (I) preferably in a quantity of 0.001 to 99.999 wt. %, more preferably of 0.1 to 99.9 wt. %, still more preferably of 0.5 to 75 wt. %, most preferably of 1.0 to 50 wt. % and in particular of 2.0 to 25 wt. %, in each case relative to the total weight of the pharmaceutical composition.

In a preferred embodiment, the pharmaceutical composition additionally contains, as well as the compounds according to the invention of the general formula (I), at least one gestagen, which is preferably selected from the group consisting of allyloestrenol, chlormadinone, cyproterone, danazol, demegestone, desogestrel, dienogest, drospirenone, dydrogesterone, ethisterone, etynodiol, gestodene, gestonorone, hydroxyprogesterone, levonorgestrel, lynestrenol, medroxyprogesterone, medrogestone, megestrol, methyloestrenol, methylnortestosterone, nomegestrol, norethisterone, norethynodrel, norgestrel, norgestimate, progesterone, promegestone, tibolone, trimegestone, 1β-hydroxytrimegestone and 6β-hydroxytrimegestone. Preferred pharmaceutically acceptable esters of the above-listed gestagens are acetates (for example chlormadinone acetate, medroxyprogesterone acetate, megestrol acetate, norethisterone acetate), caproates (for example hydroxyprogesterone caproate) and enantates (for example norethisterone enantate).

The quantity of the additional gestagen preferably corresponds to an equivalent dose of 100 to 5,000 μg, more preferably of 250 to 4,000 μg, still more preferably of 500 to 3,500 μg, most preferably of 750 to 3,000 μg and in particular of 1,000 to 2,500 μg of chlormadinone acetate.

The equivalent dose to chlormadinone acetate may be provided by an equivalent quantity of any suitable gestagen, wherein the quantity is here selected such that the gestagenic activity corresponds to that which would be brought about by the administration of the stated quantity of chlormadinone acetate. It is also possible for two or more different gestagens to be used in a quantity which overall corresponds to the stated equivalent dose. Suitable methods for determining the equivalent dose are known to a person skilled in the art.

In a preferred embodiment, the pharmaceutical composition additionally contains at least one oestrogen, which is preferably selected from the group consisting of chlorotrianisene, dienestrol, diethylstilbestrol, oestradiol(17β-oestradiol), oestriol, oestrone, ethinyl oestradiol, oestradiol benzoate, hexoestrol, mestranol, methallenoestril, methyloestrenol, promestriene and conjugated oestrogens or the pharmaceutically acceptable esters thereof, such as for example valerates, wherein a particularly preferred additional oestrogen component is ethinyl oestradiol or a combination of ethinyl oestradiol and oestradiol(17β-oestradiol).

In a preferred embodiment, the pharmaceutical composition contains a combination of at least one compound of the general formula (I) and at least one more of the above-listed gestagens and/or at least one of the above-listed oestrogens. The pharmaceutical composition particularly preferably contains a combination of at least one compound of the general formula (I) and ethinyl oestradiol or a combination of at least one compound of the general formula (I) and a combination of ethinyl oestradiol and oestradiol(17β-oestradiol).

The pharmaceutical composition may be liquid (for example a solution, dispersion, suspension or emulsion), pasty or solid (for example a powder or granular product). It is preferably solid.

The pharmaceutical composition preferably additionally contains, as well as at least one compound of the general formula (I) and optionally at least one oestrogen and/or at least a further gestagen, at least one preparation containing iron, folic acid and/or folinic acid.

Examples of preparations containing iron are iron(II) preparations, such as for example iron(II) sulfate, iron(II) carbonate, iron(II) chloride, iron(II) tartrate, iron(II) gluconate, iron(II) aspartate, iron(II) glycine sulfate, iron(II) fumarate, iron(II) ascorbate, iron(II) iodate, iron(II) succinate and ammonium iron(II) sulfate; and iron(III) preparations, such as for example iron(III) sodium citrate, iron(III) oxide/sucrose complex, sodium feredetate, iron(III) hydroxide, dextriferron, iron(III) citrate, chondroitin sulfate/iron(III) complex, iron(III) acetyltransferrin, iron(III) protein succinylate and potassium/iron(III) phosphate/citrate complex.

The folic acid or the derivative thereof is preferably present in free form or as a salt, for example as calcium folate.

In addition to at least one compound of the general formula (I), in each case optionally in the form of corresponding pharmaceutically acceptable salts and/or corresponding solvates and optionally a further gestagen and/or at least one oestrogen, the pharmaceutical composition preferably additionally contains one or more auxiliary substances, which are preferably selected from the group consisting of salt-forming agents, buffers, emulsifiers, embedding materials, thickeners, penetration promoters, film formers, binders, slip agents, surface-active substances, plasticisers, disintegration accelerators, solvents, humectants, gel formers, preservatives, stabilisers (reducing agents, antioxidants), mould release agents, fillers, lubricants, chelating agents, aroma additives, fragrances and colorants.

Suitable buffers which may be used for the pharmaceutical composition are known to a person skilled in the art. Buffers which may accordingly be used are for example succinic acid, citric acid, lactic acid, phosphoric acid, trisodium phosphate, disodium hydrogenphosphate, sodium dihydrogenphosphate, sodium carbonate, sodium hydrogencarbonate, and combinations of lactic acid with sodium hydroxide. The pH value is preferably adjusted to 2.0-5.5 with the buffer, which is preferably a mixture of citric acid and disodium hydrogenphosphate.

Emulsifiers are preferably added in quantities such that they enable uniform mixing of the components of the pharmaceutical composition according to the invention. Conventional emulsifiers preferably comprise anionic, cationic and/or nonionic surfactants. Examples of such emulsifiers preferably comprise potassium stearate, sodium stearate, ammonium stearate, triethanolamine stearate, glycerol monostearate, sodium lauryl 5-sulfate, sodium acetyl sulfate, N-(stearoyl colamino formylmethyl)pyridium, N-soya N-ethyl morpholinium ethosulfate, alkyldimethylbenzyl ammonium chloride, diisobutylphenoxyethoxyethyldimethylbenzyl ammonium chloride, acetyl pyridium chloride, monostearate, polyoxyethylene stearate, polyoxyethylene sorbitan monostearate, sorbitan, propylene glycol monostearate and/or ethoxylated lanolin. The emulsifier is preferably used in a quantity of 0.1 to 10 wt. %, relative to the total weight of the pharmaceutical composition according to the invention.

Examples of embedding materials are carnauba wax, montan glycol wax, stearic/palmitic acid, glycerol trioleate and cetylstearyl alcohol.

Thickeners which may preferably be present in the composition according to the invention comprise, for example, candelilla, carnauba and microcrystalline waxes, carbomer and polyethylene thickeners. The thickener is preferably used in a quantity of 0.5 to 2 wt. %, relative to the total weight of the pharmaceutical composition according to the invention.

Suitable penetration promoters for the purposes of the description preferably comprise penetration promoters which are selected from the group comprising acid amides and amines. Urea is particularly preferred as a penetration promoter. The penetration promoter is preferably used in a quantity of 0.5 to 10 wt. %, relative to the total weight of the pharmaceutical composition according to the invention.

Examples of film formers are shellac, methylcellulose, hydroxypropylmethylcellulose (HPMC), hydroxypropylcellulose, hydroxyethylcellulose, ethylcellulose, polyacrylates and polymethacrylates.

Binders impart cohesive properties to a pharmaceutical composition and for example improve granulation characteristics. Suitable binders are for example hydroxypropylcellulose, starch, cellulose ether, polyvinylpyrrolidone (povidone), hydroxypropylmethylcellulose, gelatin and sugars, for example sucrose and glucose syrup. The binders may constitute a proportion by weight of preferably 0.5 to 5.0 wt. % relative to the total weight of the pharmaceutical composition.

Slip agents are added to a pharmaceutical composition in order to improve its flow behaviour during granulation, to prevent adhesion of the composition to the granulation or pressing equipment, to reduce friction between the particles and to facilitate ejection of the tablets from the press moulds. Suitable slip agents are, for example, talcum, long-chain fatty acids such as stearic acid and palmitic acid, the salts thereof such as magnesium stearate and calcium stearate, polyethylene glycol and hydrogenated vegetable oils. The slip agents may constitute a proportion by weight of preferably 0.25 to 3.0 wt. % relative to the total weight of the pharmaceutical composition.

A distinction is usually drawn between slip agents and flow promoters, the latter being added to the composition after granulation and before tabletting to prevent agglomeration of the granules. One suitable flow promoter is for example colloidal silicon dioxide. The flow promoters may constitute a proportion by weight of preferably 0.1 to 3.0 wt. % relative to the total weight of the pharmaceutical composition.

Surface-active substances (surfactants) having nonionic, cationic, anionic and/or ampholytic properties may furthermore also be present. Nonionic surface-active substances are preferably used for the pharmaceutical composition, these preferably comprising sorbitan esters, such as sorbitan monolaurate, sorbitan monooleate, sorbitan monoisostearate; polyoxyethylene sorbitan esters, such as polyoxyethylene sorbitan monoisostearate, polyoxyethylene sorbitan monolaurate, polyoxyethylene sorbitan monooleate; glycerol esters, such as glycerol monoisostearate, glycerol monomyristate; polyoxyethylene glycerol ethers, such as polyoxyethylene glycerol monoisostearate, polyoxyethylene glycerol monomyristate; polyglycerol fatty acid esters, such as diglycerol monostearate, decaglycerol decaisostearate, diglycerol diisostearate; glycerol fatty acid esters, such as glycerol monocaprate, glycerol monolaurate, glycerol monomyristate, glycerol monopalmitate, glycerol monooleate, glycerol monostearate, glycerol monolinoleate, glycerol monoisostearate, glycerol monodilinoleate, glycerol monodicaprate; polyoxyethylene glycerol fatty acid esters, such as polyoxyethylene glycerol monomyristate, polyoxyethylene glycerol monooleate, polyoxyethylene glycerol monostearate; polyoxyethylene-branched alkyl ethers, such as polyoxyethylene octyl dodecyl alcohol, polyoxyethylene 2-decyl tetradecyl alcohol; polyoxyethylene alkyl ethers, such as polyoxyethylene oleyl alcohol ether, polyoxyethylene acetyl alcohol ether; polyoxyethylene-hydrogenated castor oil fatty acid esters, such as polyoxyethylene-hydrogenated castor oil, polyoxyethylene dihydrocholesterol ether, polyoxyethylene-hydrogenated castor oil isostearate and/or polyoxyethylene alkylaryl ethers, such as polyoxyethylene octylphenol ether.

Anionic surface-active substances preferably comprise salts, such as diethanolamine salt, triethanolamine salt, amino acid salt, sodium salt, potassium salt; higher fatty acids, such as oleic acid, stearic acid, isostearic acid, palmitic acid, myristic acid, ether carboxylic acid alkaline salts and N-acylamino acid salts. The surface-active substance is preferably used in a quantity of 0.1 to 10 wt. %, relative to the total weight of the pharmaceutical composition according to the invention.

Plasticisers may likewise be present in the pharmaceutical composition according to the invention. Conventional plasticisers are preferably selected from the group comprising oils and waxes, silicone oils, triglyceride esters, acetoglyceride esters, ethoxylated glycerides, alkyl esters, alkenyl esters, fatty acids, fatty alcohols, fatty alcohol esters, lanolin and the derivatives thereof, polyhydrogenated alcohols and the ethers thereof, polyhydrogenated alcohol esters, wax esters, beeswax derivatives, vegetable waxes, phospholipids, sterols and amides. The plasticisers are preferably used in a quantity of 1 to 25 wt. %, relative to the total weight of the composition according to the invention.

Disintegration accelerators (tablet disintegrants) are added to a pharmaceutical composition in order to promote disintegration of a tablet produced from the composition. Suitable tablet disintegrants are for example modified or unmodified starch (for example maize starch, wheat starch, potato starch, etc.), clay minerals, crosslinked polyvinylpyrrolidone, modified or unmodified cellulose (for example low-substituted sodium carboxymethylcellulose), gums or algins. The tablet disintegrants may constitute a proportion by weight of preferably 5.0 to 50 wt. %, more preferably of 5.0 to 15 wt. %, relative to the total weight of the pharmaceutical composition.

Solvents may also be present in the pharmaceutical composition according to the invention, for example water, ethanol, mixtures of water and ethanol, propylene glycol or glycerol. Preferably, however, the pharmaceutical composition contains no solvents, i.e. it has a (residual) moisture content of less than 10 wt. %, more preferably of less than 5.0 wt. %, still more preferably of less than 2.0 wt. %, most preferably of less than 1.0 wt. % and in particular of less than 0.5 wt. % relative to the total weight of the pharmaceutical composition.

One example of a humectant is glycerol.

Gel formers suitable for the compositions according to the invention preferably comprise natural or synthetic polymers. Natural polymers are preferably selected from the group comprising agar-agar, alginic acid, alginate, amidated pectin, propylene glycol alginate, carbomer, carrageenan, casein, dammar gum, dextrins, furcellaran, gelatin, guar gum, guar flour, gellan, gum ghatti, gum arabic, spruce sap gum, locust bean flour, karaya gum, keratin, konjac flour, L-HPC, locust bean gum, mastic, pectin, shellac, (optionally modified) starch, tara stone flour, tragacanth, xanthan gum and the derivatives thereof. Preferred synthetic polymers which may be used as gelling agents for the composition according to the invention are selected from the group comprising acrylic acid polymers, carbomer, polyacrylamides and alkylene oxide polymers. The gel formers are preferably used in a quantity of 0.1 to 5 wt. %, relative to the total weight of the composition according to the invention.

Preservatives may also be present in the pharmaceutical composition according to the invention. Examples of preservatives are alcohols (for example ethanol, chlorobutanol, phenylethyl alcohol or benzyl alcohol), acids (for example sorbic acid or benzoic acid), phenol derivatives (for example phenol, cresol or chlorocresol) or organomercury compounds such as for example phenylmercury nitrate or thiomersal. The preservatives are preferably used in a quantity of 0.001 to 15 wt. %, relative to the total weight of the pharmaceutical composition according to the invention.

Stabilisers which may be used are antioxidants and/or reducing agents. In particular, at least one reducing agent is selected for the pharmaceutical composition from the group comprising sulfites, such as sodium sulfite, potassium sulfite, ammonium sulfite, sodium hydrogensulfite, potassium hydrogensulfite, sodium bisulfite, calcium sulfate, calcium hydrogensulfite, potassium bisulfite, sodium disulfite, ammonium hydrogensulfite, sodium metabisulfite, potassium metabisulfite; mercaptocarboxylic acids, such as 2-mercaptopropionic acid, 3-mercaptopropionic acid, mercaptosuccinic acid, thioglycolic acid, ammonium thioglycolate, sodium thioglycolate, L-cysteine, dimercaptoadipic acid; mercaptoamines, such as L-cysteine ethyl ester, L-cysteine methyl ester, N-acetyl-L-cysteine, cysteamine; mercaptoamides, such as thioglycolamide, N-hydroxyethyl mercaptoacetamide, N-methyl mercaptoacetamide, 2-mercaptopropionamide; hydroxides, such as guanidine hydroxide, sodium hydroxide; alcohols and diols, such as resorcinol, thioglycerol, glycerol monothioglycolate, glycol thioglycolate; dithio compounds, such as dihydrolipoic acid, sodium dihydrolipoic acid, dithiothreitol, 1,3-dithiopropanol; lithium chloride, tris(hydroxymethyl)phosphine, thioglycol hydrazide, 2-mercaptoethanesulfonic acid, homocysteinethiolactone, polythiol polymers, salts of hydrogen sulfide, amines in alkaline solution, salts of hydrogen cyanide, borohydride, dithionite, ester salts of sulfoxylates, formic acid, oxalic acid, diazolidinyl urea, iodopropynyl butyl carbamate, chloromethylisothiazolinone, methylisothiazolinone, butylparaben, ethylparaben, methylparaben, propylparaben, isobutylparaben and phenoxyethanol. The reducing agents are preferably used in a quantity of 0.001 to 2 wt. %, relative to the total weight of the pharmaceutical composition according to the invention.

The antioxidant component used for the pharmaceutical composition is preferably at least one antioxidant selected from the group comprising ascorbic acid (vitamin C), sodium L-ascorbate, calcium L-ascorbate, ascorbyl palmitate, butylhydroxyanisole, butylhydroxytoluene, calcium disodium EDTA, propyl gallate, octyl gallate, dodecyl gallate (lauryl gallate), isoascorbic acid, sodium isoascorbate, lecithin, lactic acid, polyphosphate, sulfur dioxide, selenium, tocopherol (vitamin E), α-tocopherol, γ-tocopherol, δ-tocopherol, tin(II) chloride, citric acid, sodium citrate and potassium citrate. The antioxidant is preferably used in a quantity of 0.001 to 2 wt. %, relative to the total weight of the pharmaceutical composition according to the invention.

Fillers increase the mass and volume of a pharmaceutical composition. Suitable fillers are for example lactose, mannitol, sorbitol, cellulose, microcrystalline cellulose, xylitol, dextrose, fructose, starch, calcium carbonate (E 170), calcium phosphate, NaCaPO₄, magnesium carbonate, sucrose and mixtures thereof. The fillers may constitute a proportion by weight of preferably 70 to 95 wt. % relative to the total weight of the pharmaceutical composition.

Examples of lubricants are stearic acid, magnesium stearate, calcium stearate and zinc stearate.

Examples of chelating agents are citric acid, phenylalanine, sodium calcium edetate and disodium edetate (EDTA-Na₂).

The pharmaceutical composition may preferably contain at least one fragrance and/or a colorant. The pharmaceutical composition particularly preferably contains as fragrance or aroma additive at least one natural or nature-identical compound selected from the group comprising anethole, benzaldehyde, benzyl acetate, benzyl alcohol, benzyl formate, iso-bornyl acetate, camphene, neral, citronellal, citronellol, citronellyl acetate, para-cymene, decanal, dihydrolinalool, dihydromyrcenol, dimethylphenylcarbinol, eucalyptol, geraniol, geranyl acetate, geranyl nitrile, cis-3-hexenyl acetate, hydroxycitronellal, limonene, linalool, linalool oxide, linalyl acetate, linalyl propionate, methyl anthranilate, alpha-methylionone, methylnonylacetaldehyde, methylphenylcarbinyl acetate, menthone, iso-menthone, myrcene, myrcenyl acetate, myrcenol, nerol, neryl acetate, nonyl acetate, phenylethyl alcohol, alpha-pinene, beta-pinene, gamma-terpinene, alpha-terpinol, beta-terpinol, terpinyl acetate, para-tert.-butylcyclohexyl acetate, alpha-amylcinnamaldehyde, amyl salicylate, caryophyllene, cedrene, cinnamyl alcohol, dimethylbenzylcarbinyl acetate, ethylvanillin, eugenol, iso-eugenol, tricyclodecenyl acetate, piperonal, 3-cis-hexenyl salicylate, hexyl salicylate, lilial, gamma-methylionone, nerolidol, patchouli alcohol, phenylhexanol, beta-selinene, trichlormethylphenylcarbinyl acetate, triethyl citrate, vanillin, dimethoxybenzaldehyde, benzophenone, ethylene brassylate, galaxolide, hexylcinnamaldehyde, lyral, methyl cedryl ketone, methyl beta-naphthyl ketone, musk ketone, phenylethyl phenyl acetate, ambrettolide, cyclohexyl salicylate, delta-nonalactone, delta-undecalactone, dodecalactone, ethyl undecylenate, exaltolide, gamma-undecalactone, hexadecanolide, myristicin and musk xylene.

At least one naturally occurring mixture of fragrances or aroma additives may also be used as a fragrance or aroma additive for the pharmaceutical composition. In particular, at least one suitable fragrance or aroma additive mixture is selected from the group comprising rosemary oil, sandalwood oil, violet oil, lemon grass oil, lavender flower oil, eucalyptus oil, peppermint oil, camomile oil, clove leaf oil, cinnamon oil, thyme oil, tea tree oil, cajeput oil, niaouli oil, manuka oil, citrus oil, mountain pine oil, jasmine oil, geranium oil, caraway oil, pine-needle oil, bergamot oil, terpentine oil, linalol oil, blood orange oil, cypress oil, silver fir oil, fennel oil, grapefruit oil, ginger oil, pine-needle oil, lavandin oil, lime oil, mandarin oil, melissa oil, myrrh oil, patchouli oil, rosewood oil and thuja oil. The fragrance or aroma additive is preferably used in a quantity of 0.001 to 2 wt. %, relative to the total weight of the pharmaceutical composition according to the invention.

Colorants which are preferably used for the pharmaceutical composition comprise:
A) inorganic and organic pigments, such as for example titanium oxide, zirconium oxide, cerium oxide, zinc oxide, iron oxide, Prussian blue, carbon blacks, calcium lakes and aluminium lakes.
B) fat-soluble colorants, such as for example Sudan Red I, DC Red 17, DC Green 6, beta-carotene, soya oil, Sudan Brown, DC Yellow 11, DC Violet 2, DC Orange 5 and Quinoline Yellow.
C) water-soluble colorants, such as for example iron sulfates, (rhodamines), methylene blue and natural colorants.

The colorant is preferably used in a quantity of 0.001 to 2 wt. %, relative to the total weight of the pharmaceutical composition according to the invention.

In a preferred embodiment, the pharmaceutical composition according to the invention contains, together with at least one compound of the general formula (I) and optionally at least one oestrogen and/or at least one further gestagen, the following auxiliary substances in the following preferred quantities (percentages are relative to the total weight of the pharmaceutical composition):

| | | more | |
|---|---|---|---|
| | preferred | preferred | in particular |
| Constituent | [wt. %] | [wt. %] | [wt. %] |
| HPMC | 1.0 to 7.5 | 2.5 to 5.0 | 3.0 to 5.0 |
| Titanium | 0.1 to 2.0 | 0.5 to 1.5 | 0.7 to 1.2 |
| dioxide | | | |
| Starch | 10 to 60 | 20 to 40 | 25 to 35 |
| Lactose | 25 to 80 | 40 to 70 | 50 to 65 |
| Stearic | 0.1 to 2.5 | 0.2 to 1.5 | 0.3 to 1.0 |
| acid | | | |
| Talcum | 0.1 to 5.0 | 0.5 to 2.5 | 0.9 to 1.5 |

Production of the pharmaceutical composition according to the invention proceeds with the assistance of the conventional means, devices, methods and processes known from the prior art, such as are described for example in “Remington's Pharmaceutical Sciences”, ed. A. R. Gennaro, 17th ed., Mack Publishing Company, Easton, Pa. (1985), in particular in part 8, chapters 76 to 93.

The present invention also provides a pharmaceutical dosage form comprising the above-described pharmaceutical composition.

The pharmaceutical composition according to the invention may accordingly take the form of a liquid, semisolid or solid dosage form, for example in the form of suspensions, ointments, creams, lotions, gels, emulsions, tablets, capsules, sugar-coated tablets, powders, suppositories, dressings, vaginal rings, pessaries, implants, intrauterine pessaries, hormone spirals, sprays or depot ampoules. The dosage form according to the invention preferably assumes the form of a tablet, film tablet, sugar-coated tablet, capsule, pellet formulation, suppository, transdermal plaster or vaginal ring. Suitable embodiments are in principle known to a person skilled in the art.

If the pharmaceutical composition is formulated as a solid dosage form, the latter may also assume multiparticulate form, preferably in the form of microtablets, microcapsules, micropellets, accretion pellets, granules, extrudates, spheroids, beads or pellets, optionally packaged in capsules or press-moulded into (film) tablets, with dry-compacted forms also being possible.

If the pharmaceutical composition is formulated as a liquid or pasty dosage form, the latter may for example assume the form of a liquid, foam, cream, gel, paste, balsam, spray, ointment, lotion, rinse (conditioner), tonic, tincture, milk, purée, powder for dissolution, emulsion (oil-in-water, water-in-oil), serum, oil, shampoo, suspension, such as liposomes or nanosomes, or as a dispersion.

The dosage form according to the invention is preferably formulated for administration one, twice or three times daily, preferably for oral administration. A particularly preferred dosage form, preferably for oral administration, containing the above-described pharmaceutical composition, is one which is provided in the form of daily units.

The dosage form according to the invention may release the compound of the general formula (I) immediately (immediate release) or in controlled manner (controlled release). If release is controlled, it may be for example time-delayed (delayed release), prolonged (sustained release) or pulsed (pulsed release, repeat action release).

If the dosage form according to the invention contains auxiliary substances, these correspond to the above-listed auxiliary substances, which may also be used for formulating the pharmaceutical composition according to the invention.

In a preferred embodiment, the dosage form according to the invention, preferably in the form of daily units, preferably for oral administration, contains the compound of the general formula in a quantity of 0.001 to 99.999 wt. %, more preferably of 0.1 to 99.9 wt. %, still more preferably of 0.5 to 75 wt. %, most preferably of 1.0 to 50 wt. % and in particular of 2.0 to 25 wt. %, in each case relative to the total weight of the dosage form.

The dose of the compound of the general formula (I) contained in the dosage form according to the invention preferably amounts to at least 100 μg, more preferably to at least 200 μg, still more preferably to at least 300 μg, most preferably to at least 400 μg and in particular to at least 500 μg. In a preferred embodiment, the dose of the compound of the general formula (I) in the dosage form according to the invention is in the range from 500 μg to 3,000 μg, more preferably from 510 to 2,500 μg, still more preferably from 525 to 2,000 μg, most preferably from 550 to 1,500 μg and in particular from 600 to 900 μg. In a preferred embodiment, the compound of the general formula (I) is present in a quantity such that its dose corresponds to the equivalent dose of trimegestone in the above-defined range.

If the compound of the general formula (I) is present in combination with at least one oestrogen, preferably with ethinyl oestradiol, the dose of the oestrogen in the dosage form preferably corresponds to an equivalent dose of 5.0 to 55 μg, more preferably of 10 to 50 μg, still more preferably of 15 to 48 μg, most preferably of 20 to 45 μg and in particular of 22 to 40 μg of ethinyl oestradiol. If two or more oestrogens are present, their total dose preferably corresponds to the above-stated equivalent dose.

The equivalent dose to ethinyl oestradiol may be provided by an equivalent quantity of any suitable oestrogen, wherein the quantity is here selected such that the oestrogenic activity, preferably inhibition of ovulation, corresponds to that which would be brought about by the administration of the stated quantity of ethinyl oestradiol. It is also possible for two or more different oestrogens, for example ethinyl oestradiol in combination with oestradiol, to be used in a quantity which overall corresponds to the stated equivalent dose. Suitable methods for determining the equivalent dose are known to a person skilled in the art.

Particularly preferred embodiments for combinations of a daily dosage X of at least one compound of the general formula (I) with the daily dosages Y of ethinyl oestradiol, which may be present in the pharmaceutical dosage form according to the invention, are summarised in the following table:

| Compound of the | |
|---|---|
| general formula (I) | Ethinyl oestradiol |
| 500 ≦ X ≦ 3,000 μg | 10 μg ≦ Y ≦ 50 μg |
| 510 ≦ X ≦ 2,500 μg | 12 μg ≦ Y ≦ 48 μg |
| 525 ≦ X ≦ 2,000 μg | 15 μg ≦ Y ≦ 45 μg |
| 550 ≦ X ≦ 1,500 μg | 18 μg ≦ Y ≦ 42 μg |
| 600 ≦ X ≦ 900 μg | 20 μg ≦ Y ≦ 40 μg |

In a preferred embodiment, the compound of the general formula (I) is present in a quantity such that its dose corresponds to the equivalent dose X of trimegestone in the above-defined range.

Particularly preferred embodiments for combinations of the daily dosage X of at least one compound of the general formula (I) with the daily dosages Y of ethinyl oestradiol and the daily dosage Z of oestradiol (17β-oestradiol), which may be present in the dosage form according to the invention, are summarised in the following table:

| Compound of the | | |
|---|---|---|
| general formula (I) | Ethinyl oestradiol | Oestradiol |
| 500 ≦ X ≦ 3,000 μg | 1.0 μg ≦ Y ≦ 10 μg | 1,000 μg ≦ Z ≦ |
| | | 10,000 μg |
| 510 ≦ X ≦ 2,500 μg | 2.0 μg ≦ Y ≦ 10 μg | 1,100 μg ≦ Z ≦ |
| | | 9,000 μg |
| 525 ≦ X ≦ 2,000 μg | 3.0 μg ≦ Y ≦ 9.5 μg | 1,200 μg ≦ Z ≦ |
| | | 8,000 μg |
| 550 ≦ X ≦ 1,500 μg | 4.0 μg ≦ Y ≦ 9.5 μg | 1,300 μg ≦ Z ≦ |
| | | 7,000 μg |
| 600 ≦ X ≦ 900 μg | 5.0 μg ≦ Y ≦ 9.0 μg | 1,400 μg ≦ Z ≦ |
| | | 6,000 μg |

In a preferred embodiment, the compound of the general formula (I) is present in a quantity such that its dose corresponds to the equivalent dose X of trimegestone in the above-defined range.

The present invention also provides a cosmetic composition which contains at least one compound of the general formula (I). The cosmetic composition preferably serves for care of the skin and/or the hair, preferably by topical application.

Suitable cosmetic auxiliary substances are preferably the conventional auxiliary substances of such compositions. In this connection, reference may for example be made to the full content of H. P. Fiedler, Lexikon der Hilfsstoffe für Pharmazie, Kosmetik und angrenzende technische Gebiete, Editio Cantor Aulendorff, 2002. The above-listed auxiliary substances which may preferably be used are those which may also be present in the pharmaceutical compositions according to the invention. These auxiliary substances are physiologically acceptable and the quantities of the particular components are preferably selected such that the cosmetic composition according to the invention complies with EU Cosmetics Directive 76/768/EEC or EU Directive 95/17/EC.

Selection of the auxiliary substances and the quantities thereof to be used is determined by whether the pharmaceutical or cosmetic composition according to the invention is to be administered or applied orally, topically, subcutaneously, parenterally, intradermally, vaginally or locally, wherein oral, vaginal, subcutaneous or transdermal use is particularly preferred. Orally or percutaneously administrable preparations may also release the compound of the general formula (I) in delayed manner.

The present invention also provides a method for contraception comprising preferably oral administration of at least one compound of the general formula (I) or of the above-described pharmaceutical composition or of the above-described pharmaceutical dosage form to a woman of child-bearing age on at least 21, preferably 21 to 26, more preferably 22 to 25 and most preferably 23 or 24 successive days of a preferably 28-day menstrual cycle, beginning on day 1 of the menstrual cycle, wherein on at least one, preferably at least on 2, more preferably at least on 5, still more preferably at least on 8, most preferably at least on 14 and in particular on all of the at least 21 successive days the daily dosage of the compound of the general formula (I) is in the range from 500 μg to 3,000 μg, more preferably from 510 to 2,500 μg, still more preferably from 525 to 2,000 μg, most preferably from 550 to 1,500 μg and in particular from 600 to 900 μg.

In a preferred embodiment, the dosage of the compound of the general formula (I) amounts to a quantity such that its dose corresponds to the equivalent dose of trimegestone in the above-defined range.

In a preferred embodiment of the method according to the invention, on at least one, preferably on all of the at least 21 successive days, at least one compound of the general formula (I) is administered in combination with at least one oestrogen, wherein the oestrogen is preferably selected from the group consisting of chlorotrianisene, dienestrol, diethylstilbestrol, oestradiol(17β-oestradiol), oestriol, oestrone, ethinyl oestradiol, oestradiol benzoate, hexoestrol, mestranol, methallenoestril, methyloestrenol, promestriene and conjugated oestrogens or the pharmaceutically acceptable esters thereof, such as for example valerates. The additional oestrogen component particularly preferably comprises ethinyl oestradiol or a combination of ethinyl oestradiol and oestradiol(17β-oestradiol), wherein the quantity of the oestrogen component preferably corresponds to an equivalent dose of 5.0 to 55 μg, more preferably of 10 to 50 μg, still more preferably of 15 to 48 μg, most preferably of 20 to 45 μg and in particular of 22 to 40 μg of ethinyl oestradiol. If two or more oestrogens are used, their daily total dosage preferably corresponds to the above-stated equivalent dose.

In a particularly preferred embodiment of the method according to the invention, on none of the at least 21 successive days is an oestrogen administered without the administration of at least one compound according to the invention of the formula (I).

In a preferred embodiment of the method according to the invention, on each of the at least 21, more preferably at least 22, still more preferably at least 23, most preferably at least 24 and in particular at least 25 successive days, the daily dosage of the compound of the formula (I) is identical (=monophasic regimen), wherein administration preferably in each case proceeds in combination with at least one oestrogen.

In another preferred embodiment of the method according to the invention, the at least 21, more preferably at least 22, still more preferably at least 23, most preferably at least 24 and in particular at least 25 successive days are divided into two, three or more groups of days, wherein the daily dosage of the compound of the general formula (I) is identical on all the days within a group, but on successive days of different groups the daily dosage of the compound of the general formula (I) is different (=multiphasic regimen), and wherein administration preferably in each case proceeds in combination with at least one oestrogen.

Preferred regimens are listed in the following table, wherein the daily dose of the compound of the general formula (I) is A1, A2 or A3 and the daily dose of the at least one oestrogen is B:

| | | Number of phases | | | | | | | |
|---|---|---|---|---|---|---|---|---|---|
| | | 1 | 2 | 2 | 3 | 3 | 3 | 4 | 4 |
| Embodiment no. | | 1 | 2₁ | 2₂ | 3₁ | 3₂ | 3₃ | 4₁ | 4₂ |
| Total duration [days of 28] | | 21-25 | 21-25 | 21-25 | 21-25 | 21-25 | 21-25 | 21-25 | 21-25 |
| 1 | Duration | 21-25 | 7-13 | 7-13 | 3-8 | 3-8 | 3-8 | 3-8 | 3-8 |
| | [days] | | | | | | | | |
| | Dose of compound of formula (I) | A1 | A2 | A1 | A2 | A2 | A1 | A2 | A2 |
| | Dose of oestrogen (equivalent | B | B | B | B | B | B | B | B |
| | dose to ethinyl oestradiol) | | | | | | | | |
| 2 | Duration | | 12-18 | 12-18 | 4-15 | 4-15 | 4-15 | 4-15 | 4-15 |
| | [days] | | | | | | | | |
| | Dose of compound of formula (I) | | A1 | A2 | A2 | A1 | A2 | A1 | A2 |
| | Dose of oestrogen (equivalent | | B | B | B | B | B | B | B |
| | dose to ethinyl oestradiol) | | | | | | | | |
| 3 | Duration | | | | 4-15 | 4-15 | 4-15 | 4-15 | 4-15 |
| | [days] | | | | | | | | |
| | Dose of compound of formula (I) | | | | A1 | A2 | A2 | A2 | A1 |
| | Dose of oestrogen (equivalent | | | | B | B | B | B | B |
| | dose to ethinyl oestradiol) | | | | | | | | |
| 4 | Duration | | | | | | | 2-5 | 2-5 |
| | [days] | | | | | | | | |
| | Dose of compound of formula (I) | | | | | | | A3 | A3 |
| | Dose of oestrogen (equivalent | | | | | | | B | B |
| | dose to ethinyl oestradiol) | | | | | | | | |

The following table shows the particular value ranges of the dosages for the particular combinations of A1, A2, A3 and B for each one of these embodiments no. 1, 2₁, 2₂, 3₁, 3₂, 3₃, 4₁ and 4₂, wherein the dosage B of the at least one oestrogen is stated as an equivalent dose to ethinyl oestradiol:

| | | | more | | still more | | in | |
|---|---|---|---|---|---|---|---|---|
| | preferred | | preferred | | preferred | | particular | |
| A1 | 510-990 | μg | 525-975 | μg | 550-950 | μg | 550-750 | μg |
| A2 | 40-990 | μg | 40-750 | μg | 120-750 | μg | 260-500 | μg |
| A3 | 0-990 | μg | 0-750 | μg | 0-500 | μg | 260-500 | μg |
| B | 5.0-55 | μg | 10-50 | μg | 20-45 | μg | 25-40 | μg |

In a preferred embodiment, the compound of the general formula (I) is present in a quantity such that its dose corresponds to the equivalent dose A1, A2 or A3 of trimegestone in the above-defined range.

In a preferred embodiment of the method according to the invention, the at least one compound of the general formula (I) is not administered on all the days of the preferably 28-day menstrual cycle. Instead, it is preferred that, on the days following the at least 21 successive days,
a placebo,
a pharmaceutically acceptable preparation containing iron or
a preparation containing folic acid is administered;
or nothing at all is administered.

In this manner, it is ensured that the menstrual cycle is terminated by the withdrawal bleeding, such that a new menstrual cycle may begin. The menstrual cycle preferably lasts 28 days.

According to another preferred embodiment of the method according to the invention, it is, however, also possible for the menstrual cycle to be longer than 28 days. This may be achieved according to the invention, by the cessation of at least one compound of the general formula (I) (and optionally at least one oestrogen and/or at least one further gestagen) not occurring until a later point in time, such that the withdrawal bleeding also does not occur until a later point in time and thus the menstrual cycle also does not end until a later point in time. In this embodiment, the at least one compound of the general formula (I) is preferably administered on more than 28 successive days.

In this embodiment, (uninterrupted) administration of the at least one compound of the general formula (I) proceeds on at least 42 or 56, more preferably at least 63, still more preferably at least 84, most preferably at least 105 or 112 and in particular at least 126 or 140 successive days, such that it is not intended to initiate withdrawal bleeding within this period. According to the invention, the continuous period for which the at least one compound of the general formula (I) may be administered daily may also be still longer. In principle, it is accordingly possible to administer the at least one compound of the general formula (I) on all successive days over one or more years, without any withdrawal bleeding occurring.

In a preferred embodiment of the method according to the invention, the at least one compound of the general formula (I) is administered in combination with at least one further gestagen at least on one of the at least 21 successive days. The gestagens used in such a combination and their corresponding particular dosages correspond to those of the above-listed pharmaceutical composition according to the invention.

The method according to the invention is carried out for at least one menstrual cycle. The method according to the invention is preferably carried out for two or more, in particular for at least 6 successive menstrual cycles.

Another aspect of the invention relates to a kit comprising at least one of the above-described dosage forms according to the invention. The kit according to the invention is preferably designed for in each case once daily administration of the dosage forms contained therein.

The kit is preferably made up such that, with the assistance of the dosage forms according to the invention contained in the kit, the above-described method for contraception according to the invention may be carried out without entailing the acquisition of further dosage forms according to the invention which are not contained in the kit. The kit preferably contains one dosage form for each day, as administration preferably proceeds once daily.

If the menstrual cycle is 28 days long, the kit according to the invention preferably comprises at least as many dosage forms according to the invention as are necessary for administering at least one compound of the general formula (I) on at least 21 successive days of a 28-day menstrual cycle. If the at least one compound of the general formula (I) is administered on fewer than 28 days, for the remaining days up to the end of the 28 days of the menstrual cycle, the kit according to the invention may contain either no dosage forms at all, or preparations containing iron, preparations containing folic acid or placebos, preferably a preparation containing iron. It is necessary here for at least one of the dosage forms of the kit according to the invention to be a dosage form according to the invention.

If the menstrual cycle is extended, i.e. is more than 28 days long, the number of dosage forms contained in the kit according to the invention is correspondingly increased, wherein again at least one of the dosage forms contained is a dosage form according to the invention as described above.

In a preferred embodiment, the kit according to the invention comprises all the dosage forms which are necessary for administering at least one compound of the general formula (I) for one or two, more preferably at least three, still more preferably at least four, most preferably at least five and in particular at least six menstrual cycles.

In a preferred embodiment, the kit according to the invention is designed for mono- or multiphasic administration of at least one compound of the general formula (I) in combination with an oestrogen. The menstrual cycle is here preferably 28 days long. In the bi-, tri- and tetraphasic regimens, the daily dose of the at least one compound of the general formula (I) and of the oestrogen is in each case constant on all days within a phase and different on two successive days of different phases.

Preferred embodiments no. 1, 2₁, 2₂, 3₁, 3₂, 3₃, 4₁ and 4₂ of the kit according to the invention comprise in total 21-25 dosage forms according to the invention, wherein, depending on the number of phases, these contain the at least one compound according to the invention of the general formula (I) in dosages A1, A2, A3 and at least one oestrogen in dosage B according to the following table:

| | | Number of phases | | | | | | | |
|---|---|---|---|---|---|---|---|---|---|
| | | 1 | 2 | 2 | 3 | 3 | 3 | 4 | 4 |
| Embodiment no. | | 1 | 2₁ | 2₂ | 3₁ | 3₂ | 3₃ | 4₁ | 4₂ |
| Dosage forms, total | | 21-25 | 21-25 | 21-25 | 21-25 | 21-25 | 21-25 | 21-25 | 21-25 |
| 1 | No. of dosage units | 21-25 | 7-13 | 7-13 | 3-8 | 3-8 | 3-8 | 3-8 | 3-8 |
| | Dose of compound of formula (I) | A1 | A2 | A1 | A2 | A2 | A1 | A2 | A2 |
| | Dose of oestrogen (equivalent | B | B | B | B | B | B | B | B |
| | dose to ethinyl oestradiol) | | | | | | | | |
| 2 | No. of dosage units | | 12-18 | 12-18 | 4-15 | 4-15 | 4-15 | 4-15 | 4-15 |
| | Dose of compound of formula (I) | | A1 | A2 | A2 | A1 | A2 | A1 | A2 |
| | Dose of oestrogen (equivalent | | B | B | B | B | B | B | B |
| | dose to ethinyl oestradiol) | | | | | | | | |
| 3 | No. of dosage units | | | | 4-15 | 4-15 | 4-15 | 4-15 | 4-15 |
| | Dose of compound of formula (I) | | | | A1 | A2 | A2 | A2 | A1 |
| | Dose of oestrogen (equivalent | | | | B | B | B | B | B |
| | dose to ethinyl oestradiol) | | | | | | | | |
| 4 | No. of dosage units | | | | | | | 2-5 | 2-5 |
| | Dose of compound of formula (I) | | | | | | | A3 | A3 |
| | Dose of oestrogen (equivalent | | | | | | | B | B |
| | dose to ethinyl oestradiol) | | | | | | | | |

The following table shows the particular value ranges of the dosages for the particular combinations of A1, A2, A3 and B for each one of these embodiments no. 1, 2₁, 2₂, 3₁, 3₂, 3₃, 4₁ and 4₂, wherein the dosage B of the at least one oestrogen is stated as an equivalent dose to ethinyl oestradiol:

| | | | more | | still more | | in | |
|---|---|---|---|---|---|---|---|---|
| | preferred | | preferred | | preferred | | particular | |
| A1 | 510-990 | μg | 525-975 | μg | 550-950 | μg | 550-750 | μg |
| A2 | 40-990 | μg | 40-750 | μg | 120-750 | μg | 260-500 | μg |
| A3 | 0-990 | μg | 0-750 | μg | 0-500 | μg | 260-500 | μg |
| B | 5.0-55 | μg | 10-50 | μg | 20-45 | μg | 25-40 | μg |

In a preferred embodiment, the compound of the general formula (I) is present in a quantity such that its dose corresponds to the equivalent dose A1, A2 or A3 of trimegestone in the above-defined range.

The compound of the general formula (I) is preferably used in combination with ethinyl oestradiol or in combination with ethinyl oestradiol and oestradiol (17β-oestradiol).

In the bi-, tri- and tetraphasic regimens, the daily dose of the compound of the general formula (I) and of the oestrogen is in each case constant on all days within a phase and different on two successive days of different phases.

At least one compound of the general formula (I), optionally in combination with an oestrogen and/or a further gestagen, may also be taken optionally for a period of more than 28 days for therapeutic reasons, such as for example for the treatment and/or prevention of at least one of the complaints or diseases selected from the group consisting of rosacea; psoriasis; bleeding disorders; dysmenorrhoea (painful periods); diseases dependent on the menstrual cycle, such as endometriosis, polycystic ovarian syndrome (PCOS), uterus myomatosus, functional cysts, mood swings dependent on the menstrual cycle, premenstrual dysphoric disorder (PMDD), premenstrual syndrome (PMS) and headache/migraine; diseases influenced by the menstrual cycle, such as epilepsy, multiple sclerosis, diabetes mellitus, depression, schizophrenia, asthma and Parkinson's disease; and androgen-induced disorders, such as seborrhoea, acne, androgenetic alopecia and hirsutism.

The present invention accordingly also provides the treatment and/or prevention of at least one of the complaints or diseases selected from the group consisting of rosacea; psoriasis; bleeding disorders; dysmenorrhoea (painful periods); diseases dependent on the menstrual cycle, such as endometriosis, polycystic ovarian syndrome (PCOS), uterus myomatosus, functional cysts, mood swings dependent on the menstrual cycle, premenstrual dysphoric disorder (PMDD), premenstrual syndrome (PMS) and headache/migraine; diseases influenced by the menstrual cycle, such as epilepsy, multiple sclerosis, diabetes mellitus, depression, schizophrenia, asthma and Parkinson's disease; and androgen-induced disorders, such as seborrhoea, acne, androgenetic alopecia and hirsutism by the pharmaceutical composition or dosage form according to the invention, which is in particular also suitable for contraception.

The present invention also provides the use of a compound of the general formula (I) as a medicine.

The present invention also provides the use of at least one compound of the general formula (I), optionally in combination with at least one oestrogen and/or a further gestagen, for the production of a dosage form as described above for hormone replacement therapy (HRT).

The present invention also provides the use of at least one compound of the general formula (I), optionally in combination with at least one oestrogen and/or a further gestagen, for the production of a dosage form as described above for the treatment and/or prevention of at least one of the complaints or diseases selected from the group consisting of rosacea; psoriasis; bleeding disorders; dysmenorrhoea (painful periods); diseases dependent on the menstrual cycle, such as endometriosis, polycystic ovarian syndrome (PCOS), uterus myomatosus, functional cysts, mood swings dependent on the menstrual cycle, premenstrual dysphoric disorder (PMDD), premenstrual syndrome (PMS) and headache/migraine; diseases influenced by the menstrual cycle, such as epilepsy, multiple sclerosis, diabetes mellitus, depression, schizophrenia, asthma and Parkinson's disease; and androgen-induced disorders, such as seborrhoea, acne, androgenetic alopecia and hirsutism.

The following Examples serve to illustrate the invention, but should not be interpreted as limiting.

Trimegestone Sulfate, Pyridinium Salt:

Synthesis was carried out on the basis of the method described in DE 1 152 105 for 17α-hydroxyprogesterone. Trimegestone sulfate, pyridinium salt was produced directly from trimegestone (1) by reaction with sulfur trioxide (as pyridine complex; (2)) in pyridine:

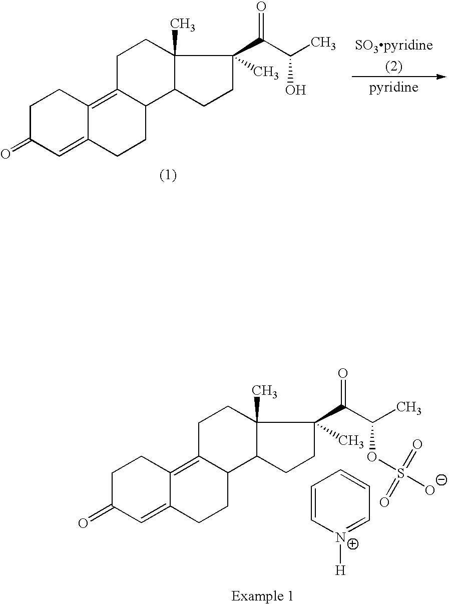

The sulfur trioxide pyridine complex and pyridine reagents were purchased from Sigma-Aldrich and used in the synthesis without further purification.

Method:

Trimegestone (3.18 g; 9.29 mmol) and sulfur trioxide pyridine complex (1.63 g; 10.24 mmol) were dissolved in pyridine (16.5 mL). The batch was stirred for two hours at room temperature. The reaction monitoring then carried out by thin-layer chromatography (silica gel; ethyl acetate/n-hexane/methanol: 2/2/1) revealed complete conversion. The reaction mixture was combined with 0.1 mL of water and stirred for 30 minutes. The reaction mixture was then combined with 165 mL of diethyl ether and stirred for a further two hours at room temperature. The precipitated solid was suction filtered out with a sintered-glass filter, thoroughly washed with diethyl ether and dried initially in air (approx. 1 hour) and then under a high vacuum (4 hours).

Yield: 4.51 g (97.0%) of beige-coloured solid, [α]_{D}=−166.1 (c=1.0; MeOH)

¹H-NMR (400 MHz, DMSO-d₆)j

b=0.77 (s, 3H); 1.11 (s, 3H); 1.09-1.39 (m, 3H); 1.28 (d, J=6.5 Hz, 3H); 1.48-1.60 (m, 2H); 1.62-1.73 (m, 1H); 1.81-1.89 (m, 1H); 2.01-2.18 (m, 2H); 2.20-2.59 (m, 7H); 2.78 (dd, J=15.6/3.5 Hz, 1H); 2.89 (dt, J=14.6/5.0 Hz, 1H); 4.93 (q, J=6.5 Hz, 1H); 5.57 (s, 1H); 8.12 (dd, J=7.5/6.8 Hz, 2H); 8.65 (dt, J=7.5/1.5 Hz, 1H); 8.96 (dd, J=6.5/1.5 Hz, 2H) ppm.

¹³C-NMR (100 MHz, DMSO-d₆h

δ=14.94; 18.33; 20.98; 23.46; 25.04; 25.17; 27.18; 30.12; 30.60; 31.91; 36.63; 38.85; 44.63; 50.34; 59.93; 72.71; 121.25; 124.55; 127.16; 142.24; 146.18; 156.57; 197.83; 211.16 ppm.

The relative affinity of trimegestone sulfate for the human progesterone receptor was determined experimentally and compared with the 6β—OH and the 1β—OH metabolites of trimegestone (cf. EP-A 808 845). The results are summarised in the following table:

| | Relative binding affinity to |
|---|---|
| Compound | progesterone receptor |
| Progesterone | ≡100 |
| 6β-OH trimegestone | 12 |
| 1β-OH trimegestone | 64 |
| Trimegestone | 40 |
| sulfate | |

As the data demonstrate, the trimegestone sulfate according to the invention exhibits a relative affinity for the progesterone receptor of 40% of the affinity of natural progesterone.

McPhail Test

Trimegestone sulfate was tested in the McPhail test, which is a test for progesterone and like substances. Immature female rabbits are treated with 150 IU of estrone over a period of 6 days. The test material is then given in five daily subcutaneous doses. Progestational proliferation of the endometrium is noted and the results estimated according to a scale from 0 to 4.0. The amount required to produce an average response is taken as a unit, equivalent to 0.25 mg of progesterone.

As demonstrated by the experimental results summarized in the table here below, trimegestone sulfate exerts dose-dependent potent endometrium transforming effects in the McPhail test, which is specific for progestational effects:

| Group | Mean McPhail |
|---|---|
| (dose: mg/kg/day s.c.) | Score (max. 4.0) |
| Progesterone | |

| | | |
|---|---|---|
| 0.1 | mg | 2.25 |
| 1.0 | mg | 3.75 |

Trimegestone sulfate

| | | |
|---|---|---|
| 0.0001 | mg | 0.75 |
| 0.001 | mg | 0.88 |
| 0.01 | mg | 2.1 |
| 0.1 | mg | 3.31 |
| 1.0 | mg | 2.93 |
| 10.0 | mg | 3.68 |

1. A compound of the formula (I) 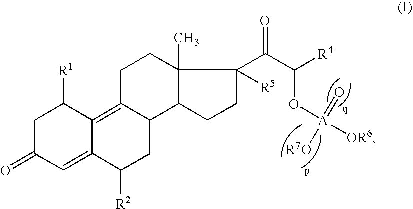
in which
A is either a sulfur atom, p=0 and q=1 or 2; or is a phosphorus atom and p=1 and q=0 or 1;
R¹ and R² are in each case mutually independently —H, —OH or —OCO—R³;
R³, R⁴, R⁵, R⁶ and R⁷ in each case mutually independently are in each case —H or a linear or branched hydrocarbon residue with 1 to 12 carbon atoms, wherein the hydrocarbon residue is unsubstituted or substituted with optionally 1, 2, 3, 4 or 5 substituents in each case mutually independently selected from the group consisting of —F, —Cl, —Br, —I and —OH;
or a pharmaceutically acceptable salt and/or solvate thereof.

2. A compound according to claim 1, wherein
A is a sulfur atom with p=0 and q=2;
R¹ and R² are in each case mutually independently —H, —OH or —OCO—R³; and
R⁴ and R⁵ are in each case mutually independently —C₁₋₆ alkyl;
or a pharmaceutically acceptable salt and/or solvate thereof.

3. A compound according to claim 1 having the formula (I-A) 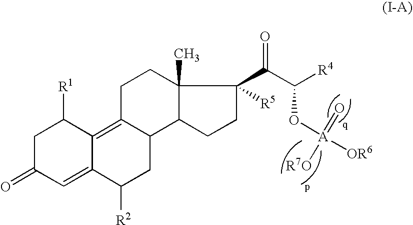
or a pharmaceutically acceptable salt and/or solvate thereof.

4. A compound according to claim 1 wherein R¹ and/or R² is —H; or a pharmaceutically acceptable salt and/or solvate thereof.

5. A compound according to claim 1 wherein R⁴ and/or R⁵ is —C₁₋₆ alkyl; or a pharmaceutically acceptable salt and/or solvate thereof.

6. A compound according to claim 1 wherein R¹ is —H and R² is —OH or —OCO—C₁₋₆ alkyl; or R¹ is —OH or —OCO—C₁₋₆ alkyl and R² is —H; or a pharmaceutically acceptable salt and/or solvate thereof.

7. A compound according to claim 1 wherein the compound is selected from the group consisting of 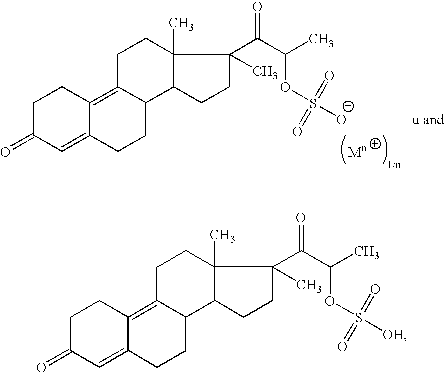
wherein Mⁿ⁺ is a pharmaceutically acceptable cation and n is 1, 2 or 3.

8. A method for the production of the compound of claim 1 comprising the step
(a) reacting a compound of the general formula (II) 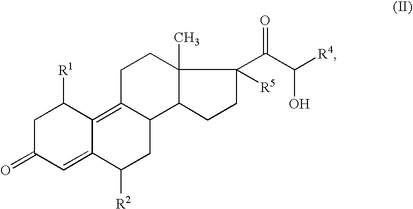
wherein R¹, R², R⁴ and R⁵ in each case have the meaning as defined in claim 1,
with a halide or anhydride of sulfurous acid, of sulfuric acid, of phosphorous acid or of phosphoric acid.

9. A method according to claim 8 which comprises reacting the compound of formula II with an anhydride of sulfuric acid, wherein the anhydride of sulfuric acid is complexed with pyridine.

10. A pharmaceutical composition comprising a contraceptive effective amount of a compound according to claim 1 or a pharmaceutically acceptable salt and/or solvate thereof.

11. A pharmaceutical composition according to claim 10 wherein the pharmaceutical composition additionally contains an oestrogen.

12. A pharmaceutical composition according to claim 11, wherein the oestrogen is ethinyl oestradiol.

13. A pharmaceutical composition according to claim 12 wherein the quantity of oestrogen corresponds to an equivalent dose of 5.0 to 55 μg of ethinyl oestradiol.

14. A composition according to claim 10 additionally comprising one or more auxiliary substances selected from the group consisting of salt-forming agents, buffers, emulsifiers, embedding materials, thickeners, penetration promoters, film formers, binders, slip agents, surface-active substances, plasticisers, disintegration accelerators, solvents, humectants, gel formers, preservatives, stabilisers, mold release agents, fillers, lubricants, chelating agents, aroma additives, fragrances and colorants.

15. A dosage form comprising a pharmaceutical composition according to claim 10.

16. A dosage form according to claim 15 wherein the dosage form is formulated for once daily administration.

17. A dosage form according to claim 15 wherein the dosage form is formulated for oral administration.

18. A method for contraception comprising administering a dosage form according to claim 15 to women of child-bearing age on at least 21 successive days, beginning on day 1 of the menstrual cycle.

19. A method according to claim 18 wherein the administration proceeds orally.
